(19) United States
(12) Plant Patent Application Publication
(10) Pub. No.: US 2013/0081187 P1
(43) Pub. Date: Mar. 28, 2013

(54) Tiarella plant named 'Sunset Ridge'

(50) ;

(71) Applicant: Janet N. Egger, Wilsonville, OR (US)

(72) Inventor: Janet N. Egger, Wilsonville, OR (US)

(73) Assignee: Terra Nova Nurseries, Inc.

(21) Appl. No.: 13/200,476

(22) Filed: Sep. 22, 2011

(51) Int. Cl. A01H 5/00

(52) U.S. Cl. PLT/486

(57) ABSTRACT

A new and distinct cultivar of Tiarella plant characterized by glossy leaves, large, ovate leaves with prominent dark markings along the veins and in splatters away from the veins, an excellent trailing habit, and excellent vigor.

### BOTANICAL DENOMINATION

Tiarella spp.

### VARIETY DESIGNATION

‘Sunset Ridge’

### BACKGROUND OF THE INVENTION

The present invention relates to a new and distinct cultivar of a hardy herbaceous perennial of the genus Tiarella, and known by the cultivar name ‘Sunset Ridge’. The genus Tiarella is a member of the family Saxifragaceae.

The new cultivar originated from a planned breeding program as a cross between Tiarella 32-01 as the seed parent and Tiarella 37-09, as the pollen parent. Both parents are proprietary, unreleased Tiarella.

Compared to Tiarella ‘Appalachian Trail’ (U.S. Plant patent application No. 12/927,595), the new cultivar has leaves that are glossy and larger.

### SUMMARY OF THE INVENTION

This plant is uniquely characterized by the following:

1. glossy leaves,

2. large, ovate leaves with prominent dark markings along the veins and in splatters away from the veins,

3. an excellent trailing habit,

4. and excellent vigor.

The new variety has been reproduced only by asexual propagation (cuttings and micropropagation). Each of the progeny exhibits identical characteristics to the original plant. Asexual propagation by cuttings and micropropagation using standard techniques with terminal and lateral shoots as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows a close up of the foliage of Tiarella ‘Sunset Ridge’ in July from plants grown indoors.

FIG. 2 shows Tiarella ‘Sunset Ridge’ growing outside in the garden in a 10″ hanging basket in August in Canby, Oreg.

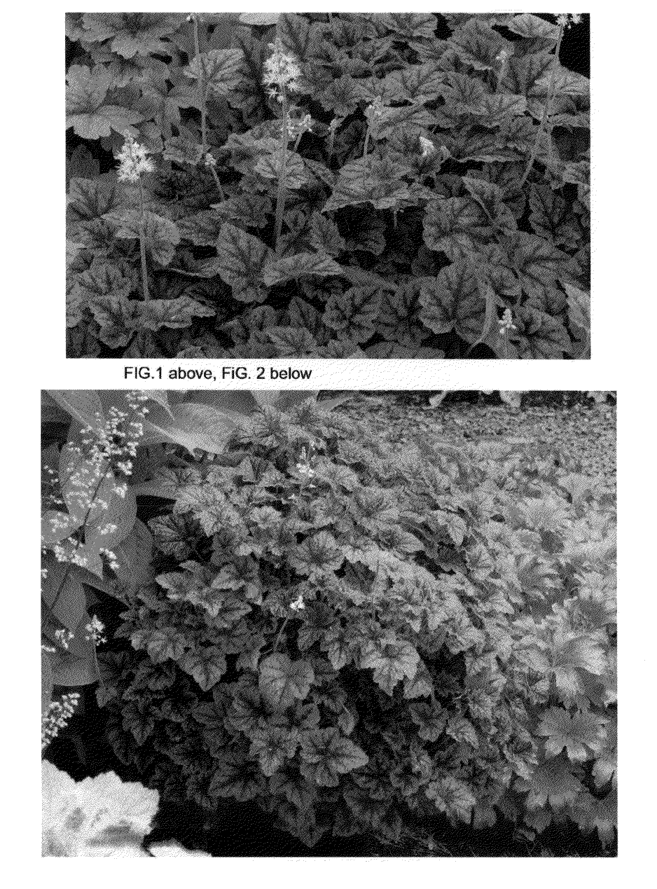

### DETAILED PLANT DESCRIPTION

The following is a detailed description of the new Tiarella cultivar based on observations of 6-month-old plants grown in one gallon containers in a warm greenhouse in April in Canby, Oreg. Canby is Zone 8 on the USDA Hardiness map. Temperatures range from a high of 95 degrees F. in August to an average low of 32 degrees F. in January. Normal rainfall in Canby is 42.8 inches per year. The color descriptions are all based on The Royal Horticultural Society Colour Chart, 5^{th} edition.
Plant:
   Type.—herbaceous perennial.
   Hardiness.—USDA Zones 4 to 9.
   Form.—trailing.
   Size.—38 cm wide and 16 cm high from the soil to the top of the foliage.
   Vigor.—excellent.
   Roots.—fibrous, freely branching, fine, and white in color; roots develop easily from stem cuttings.
Stem:
   Type.—decumbent with adventitious roots at the nodes.
   Size.—grows to 3 mm wide and 30 cm long.
   Surface.—pubescent.
   Internodes.—1.5 cm to 3 cm long.
   Color.—closest to Brown 200C.
Leaves:
   Type.—simple.
   Arrangement.—rosette.
   Shape.—broadly ovate.
   Lobing.—5 shallow lobes, terminal lobe the longest, all with secondary lobes.
   Margins.—crenate.
   Venation.—palmate.
   Apex.—acute.
   Base.—cordate, strongly overlapping.
   Blade.—grows to 13 cm long and 11.5 cm wide.
   Surface.—hispid top and bottom, glossy.
   Petiole description.—7 to 12 cm long and 2.5 mm wide, hirsute, Grey Brown N199B.
   Leaf color.—topside, Green 137A with the leaf center pattern Greyed Purple N187A; bottom side Yellow Green 147B with markings tinted Greyed Purple N187A.
Inflorescence:
   Type.—raceme.
   Flower number.—about 30 per raceme.
   Size of inflorescences.—grows to 11.5 cm long and 2.5 cm wide.
   Number of inflorescences.—8 in first spring flush.
   Bloom period.—March to mid June in Canby, Oreg.
   Peduncle.—unbranched, grows to 21 cm long and 2 mm wide at the base, hispid, Brown N199A.
   Pedicel.—6 mm long and 1 mm wide, hispid, Brown N199A.
   Lastingness.—a raceme blooms for about 3 weeks.
Flower bud:
   Size.—4 mm deep and 2.5 mm wide.
   Description.—ovoid, down facing until open.
   Surface.—glandular.
   Color.—light pink, White N155B.
Flower:
   Type.—perfect, actinomorphic, sepals petaloid.
   Shape.—rotate.
   Flower size.—7 mm wide and 6 mm deep including stamens and pistil.
   Corolla.—5 petals, each 3 mm long and 1 mm wide, lanceolate with a clawed base, margin entire, tip acuminate, glabrous on both sides, White NN155D inside and outside.
   Calyx.—6 mm wide and 1.5 mm deep, 5 sepals, backs glandular, inside glabrous, petaloid, parted almost to the base; lobes obovate, tips obtuse, margins entire, 2 mm long and 1.5 mm wide, White NN155C.
   Stamens.—10, conspicuously exerted; filaments 5 mm long, White NN155D; anthers undehisced Orange Red 31D; pollen Yellow Orange 22B.
   Pistil.—1, White N155B, 5 mm long and 2 mm wide, ovary 2 mm long and 2 mm wide, glandular.
   Fragrance.—none.
Fruit and seed: 6 mm long and 2.5 mm wide, Yellow Green 146C with little seed set; seed Brown 200A, oval, 1.5 mm long
Pest and disease resistance: This new hybrid shows good mildew tolerance, the main problem for Tiarella. No major pest problems. It is susceptible to root weevils, like all Tiarella.

I claim:

1. A new and distinct cultivar of Tiarella plant herein illustrated and described.
